(19) United States
(12) Patent Application Publication
(10) Pub. No.: US 2017/0128717 A1
(43) Pub. Date: May 11, 2017

(54) ELECTRODE ASSEMBLY FOR A COCHLEAR LEAD THAT INHIBITS TWISTING

(71) Applicant: ADVANCED BIONICS AG, STAEFA (CH)

(72) Inventors: Mark B. Downing, Valencia, CA (US); Kate Purnell, Valencia, CA (US)

(73) Assignee: ADVANCED BIONICS AG, STAEFA (CH)

(21) Appl. No.: 15/318,655

(22) PCT Filed: Jun. 17, 2014
(86) PCT No.: PCT/US2014/042779

§ 371 Date: Dec. 13, 2016

(51) Int. Cl. A61N 1/05

(52) U.S. Cl. CPC A61N 1/0541

(57) ABSTRACT

An electrode assembly for a cochlear lead is configured to stimulate an auditory nerve from within a cochlea. The electrode assembly includes a conductive support structure for supporting an electrode and having two wings that are folded toward each other to form a wire carrier for a bundle of wires of the cochlear lead; and at least one of the wings comprising a tab extending from that wing along a longitudinal axis of the cochlear lead to inhibit twisting of the cochlear lead.

### BACKGROUND

In human hearing, hair cells in the cochlea respond to sound waves and produce corresponding auditory nerve impulses. These nerve impulses are then conducted to the brain and perceived as sound.

Damage to the hair cells results in loss of hearing because sound energy which is received by the cochlea is not transduced into auditory nerve impulses. This type of hearing loss is called sensorineural deafness. To overcome sensorineural deafness, cochlear implant systems, or cochlear prostheses, have been developed. These cochlear implant systems bypass the defective or missing hair cells located in the cochlea by presenting electrical stimulation directly to the ganglion cells in the cochlea. This electrical stimulation is supplied by an electrode array which is implanted in the cochlea. The ganglion cells then generate nerve impulses which are transmitted through the auditory nerve to the brain. This leads to the perception of sound in the brain and provides at least partial restoration of hearing function.

### BRIEF DESCRIPTION OF THE DRAWINGS

The accompanying drawings illustrate various examples of the principles described herein and are a part of the specification. The illustrated examples are merely examples and do not limit the scope of the claims.

FIG. 1 is a diagram showing an illustrative cochlear implant system in use, according to one example of principles described herein.

FIG. 2 is a diagram showing external components of an illustrative cochlear implant system, according to one example of principles described herein.

FIG. 3 is a diagram showing the internal components of an illustrative cochlear implant system, according to one example of principles described herein.

FIG. 4 is a perspective view of an illustrative electrode array being inserted into a cochlea, according to one example of principles described herein.

FIG. 5 is a top view of a patterned sheet of electrochemically activated material attached to a sacrificial substrate, according to one example of principles described herein.

FIG. 6 is a top view of a patterned sheet of flexible conductive material which is attached to underlying electrode pads, according to one example of principles described herein.

FIGS. 7A and 7B are a perspective and cross-sectional view, respectively, of one illustrative example of a composite electrode assembly having an integral wire carrier, according to one example of principles described herein.

FIG. 7C is a cross-sectional view of another illustrative example of a composite electrode assembly having an integral wire carrier, according to one example of principles described herein.

FIG. 8 is a top view of a patterned sheet of flexible conductive material which is attached to underlying electrode pads, according to another example of principles described herein.

FIG. 9 is a perspective view of one illustrative example of a composite electrode assembly having an integral wire carrier that resists twisting, according to one example of principles described herein.

FIG. 10 is a top view of another illustrative example of a composite electrode assembly having an integral wire carrier that resists twisting, according to one example of principles described herein.

FIG. 11 is a top view of another illustrative example of a composite electrode assembly having an integral wire carrier that resists twisting, according to one example of principles described herein.

FIG. 12 is a top view of another illustrative example of a composite electrode assembly having an integral wire carrier that resists twisting, according to one example of principles described herein.

FIG. 13 is a top view of another illustrative example of a composite electrode assembly having an integral wire carrier that resists twisting, according to one example of principles described herein.

FIG. 14 is a top view of another illustrative example of a composite electrode assembly having an integral wire carrier that resists twisting, according to one example of principles described herein.

FIG. 15 is a perspective view of one illustrative example of a composite electrode assembly having an integral wire carrier that resists twisting, according to one example of principles described herein.

FIG. 16 is a flowchart showing one illustrative method for forming an electrode in a cochlear electrode array, according to one example of principles described herein.

Throughout the drawings, identical reference numbers designate similar, but not necessarily identical, elements.

### DETAILED DESCRIPTION

As mentioned above, individuals with hearing loss can be assisted by a number of hearing devices, including cochlear implants. Cochlear implants are made up of both external and implanted components. The external components detect environmental sounds and convert the sounds into acoustic signals. These acoustic signals are separated into a number of parallel channels of information, each representing a narrow band of frequencies within the perceived audio spectrum. Ideally, each channel of information should be conveyed selectively to a subset of auditory nerve cells that normally transmit information about that frequency band to the brain. Those nerve cells are arranged in an orderly tonotopic sequence, from the highest frequencies at the basal end of the cochlear spiral to progressively lower frequencies towards the apex. An electrode array is inserted into the cochlea and has a number of electrodes which corresponded to the tonotopic organization of the cochlea. Electrical signals are transmitted through a wire to each of the electrodes in the electrical array. When an electrode is energized, it transfers the electrical charge to the surrounding fluids and tissues. This triggers the ganglion cells to generate nerve impulses which are conveyed through the auditory nerve to the brain and perceived as sound.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “an example,” “an example,” or similar language means that a particular feature, structure, or characteristic described in connection with the example or example is included in at least that one example, but not necessarily in other examples. The various instances of the phrase “in one example” or similar phrases in various places in the specification are not necessarily all referring to the same example.

A cochlear electrode array is a thin, elongated, flexible carrier containing several longitudinally disposed and separately connected stimulating electrode contacts, conventionally numbering about 6 to 30. According to one illustrative example, the electrode array may be constructed out of biocompatible silicone, platinum-iridium wires, and platinum electrodes. This gives the distal portion of the lead the flexibility to curve around the helical interior of the cochlea.

To place the electrode array into the cochlea, the electrode array may be inserted through a cochleostomy or via a surgical opening made in the round window of the cochlea. The electrode array is inserted through the opening into the scala tympani, one of the three parallel ducts that make up the spiral-shaped cochlea. The electrode array is typically inserted into the scala tympani duct in the cochlea to a depth of about 13 to 30 mm.

In use, the cochlear electrode array delivers electrical current into the fluids and tissues immediately surrounding the individual electrode contacts to create transient potential gradients that, if sufficiently strong, cause the nearby auditory nerve fibers to generate action potentials. The auditory nerve fibers branch from cell bodies located in the spiral ganglion, which lies in the modiolus, adjacent to the inside wall of the scala tympani. The density of electrical current flowing through volume conductors such as tissues and fluids tends to be highest near the electrode contact that is the source of such current. Consequently, stimulation at one contact site tends to selectively activate those spiral ganglion cells and their auditory nerve fibers that are closest to that contact site.

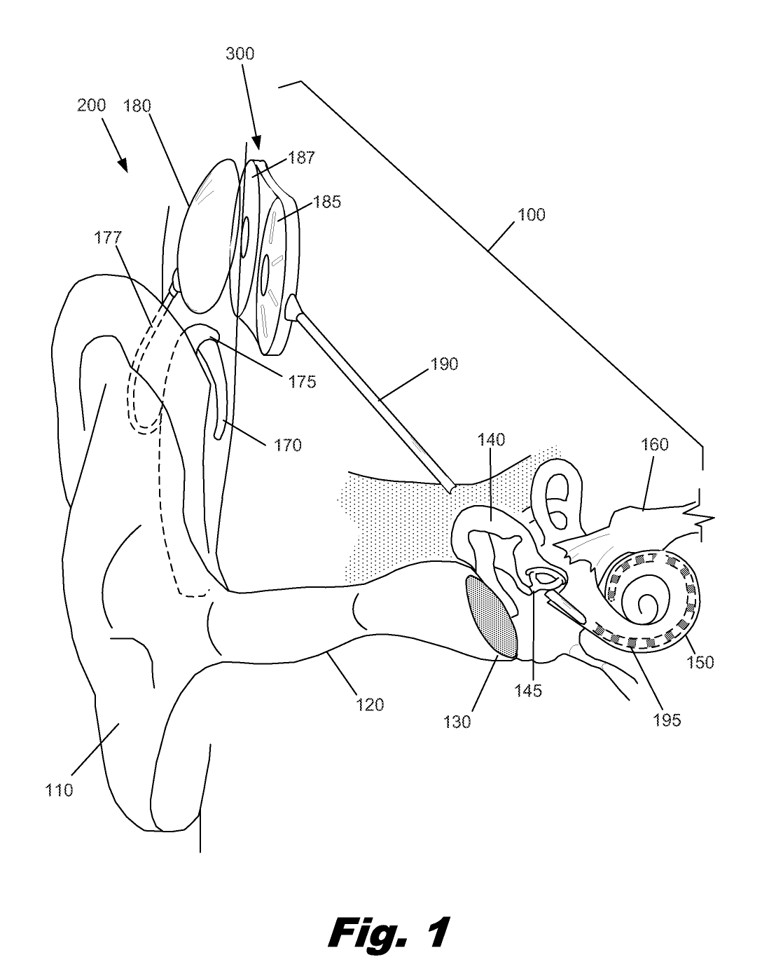

FIG. 1 is a diagram showing one illustrative example of a cochlear implant system (100) having a cochlear implant (300) with an electrode array (195) that is surgically placed within the patient's auditory system. Ordinarily, sound enters the external ear, or pinna, (110) and is directed into the auditory canal (120) where the sound wave vibrates the tympanic membrane (130). The motion of the tympanic membrane is amplified and transmitted through the ossicular chain (140), which consists of three bones in the middle ear. The third bone of the ossicular chain (140), the stirrup (145), contacts the outer surface of the cochlea (150) and causes movement of the fluid within the cochlea. Cochlear hair cells respond to the fluid-borne vibration in the cochlea (150) and trigger neural electrical signals that are conducted from the cochlea to the auditory cortex by the auditory nerve (160).

As indicated above, the cochlear implant (300) is a surgically implanted electronic device that provides a sense of sound to a person who is profoundly deaf or severely hard of hearing. In many cases, deafness is caused by the absence or destruction of the hair cells in the cochlea, i.e., sensorineural hearing loss. In the absence of properly functioning hair cells, there is no way auditory nerve impulses can be directly generated from ambient sound. Thus, conventional hearing aids, which amplify external sound waves, provide no benefit to persons suffering from complete sensorineural hearing loss.

As discussed above, the cochlear implant (300) does not amplify sound, but works by directly stimulating the auditory nerve (160) with electrical impulses representing the ambient acoustic sound. Cochlear prosthesis typically involves the implantation of electrodes into the cochlea. The cochlear implant operates by direct electrical stimulation of the auditory nerve cells, bypassing the defective cochlear hair cells that normally transduce acoustic energy into electrical energy.

External components (200) of the cochlear implant system can include a Behind-The-Ear (BTE) unit (175), which contains the sound processor and has a microphone (170), a cable (177), and a transmitter (180). The microphone (170) picks up sound from the environment and converts it into electrical impulses. The sound processor within the BTE unit (175) selectively filters and manipulates the electrical impulses and sends the processed electrical signals through the cable (177) to the transmitter (180). The transmitter (180) receives the processed electrical signals from the processor and transmits them to the implanted antenna (187) by electromagnetic transmission. In some cochlear implant systems, the transmitter (180) is held in place by magnetic interaction with a magnet in the center of the underlying antenna (187).

The components of the cochlear implant (300) include an internal processor (185), an antenna (187), and a cochlear lead (190) which terminates in an electrode array (195). The internal processor (185) and antenna (187) are secured beneath the user's skin, typically above and behind the pinna (110). The antenna (187) receives signals and power from the transmitter (180). The internal processor (185) receives these signals and performs one or more operations on the signals to generate modified signals. These modified signals are then sent along a number of delicate wires which pass through the cochlear lead (190). These wires are individually connected to the electrodes in the electrode array (195). The electrode array (195) is implanted within the cochlea (150) and provides electrical stimulation to the auditory nerve (160).

The cochlear implant (300) stimulates different portions of the cochlea (150) according to the frequencies detected by the microphone (170), just as a normal functioning ear would experience stimulation at different portions of the cochlea depending on the frequency of sound vibrating the liquid within the cochlea (150). This allows the brain to interpret the frequency of the sound as if the hair cells of the basilar membrane were functioning properly.

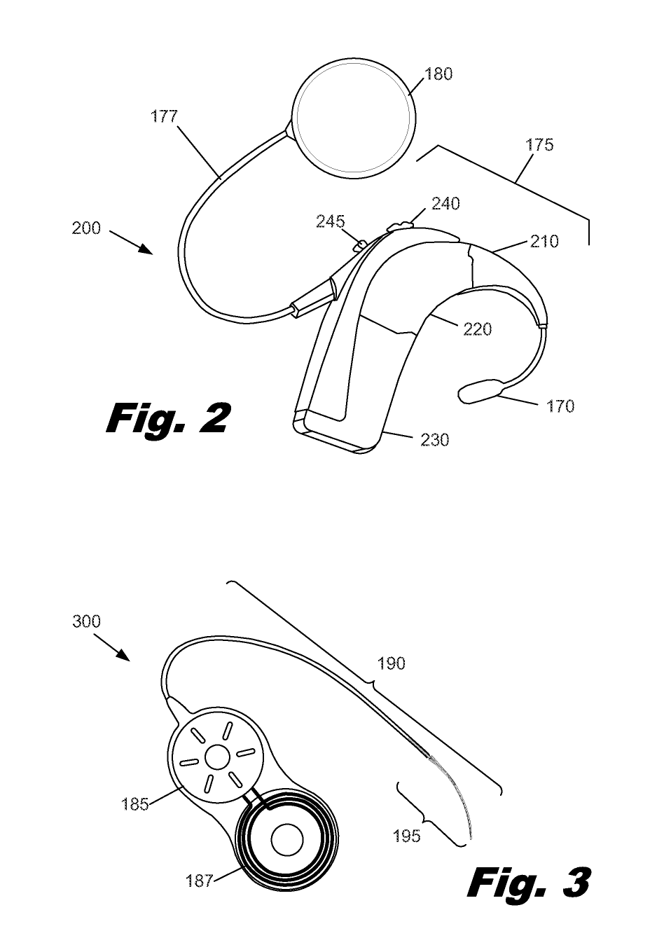

FIG. 2 is an illustrative diagram showing a more detailed view of the external components (200) of one example of a cochlear implant system. External components (200) of the cochlear implant system include a BTE unit (175), which comprises a microphone (170), an ear hook (210), a sound processor (220), and a battery (230), which may be rechargeable. The microphone (170) picks up sound from the environment and converts it into electrical impulses. As discussed above, the sound processor (220) selectively filters and manipulates the electrical impulses and sends the processed electrical signals through a cable (177) to the transmitter (180). A number of controls (240, 245) adjust the operation of the processor (220). These controls may include a volume switch (240) and program selection switch (245). The transmitter (180) receives the processed electrical signals from the processor (220) and transmits these electrical signals and power from the battery (230) to the cochlear implant by electromagnetic transmission.

FIG. 3 is an illustrative diagram showing one example of a cochlear implant (300), including an internal processor (185), an antenna (187), and a cochlear lead (190) having an electrode array (195). The cochlear implant (300) is surgically implanted such that the electrode array (195) is internal to the cochlea, as shown in FIG. 1. The internal processor (185) and antenna (187) are secured beneath the user's skin, typically above and behind the pinna (110, FIG. 1), with the cochlear lead (190) connecting the internal processor (185) to the electrode array (195) within the cochlea. As discussed above, the antenna (187) receives signals from the transmitter (180) and sends the signals to the internal processor (185). The internal processor (185) modifies the signals and passes them along the appropriate wires to activate one or more of the electrodes within the electrode array (195). This provides the user with sensory input that is a representation of external sound waves sensed by the microphone (170).

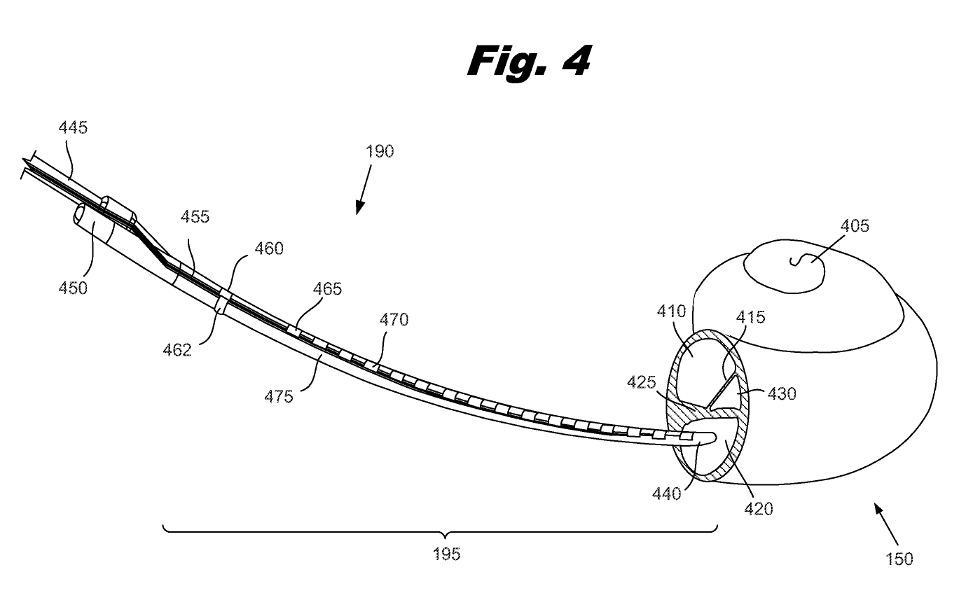

FIG. 4 is a partially cut away perspective view of a cochlea (150) and shows an illustrative electrode array (195) being inserted into the cochlea (150). The primary structure of the cochlea is a hollow, helically coiled, tubular bone, similar to a nautilus shell. The coiled tube is divided through most of its length into three fluid-filled spaces (scalae). The scala vestibuli (410) is partitioned from the scala media (430) by Reissner's membrane (415) and lies superior to it. The scala tympani (420) is partitioned from the scala media (430) by the basilar membrane (425) and lies inferior to it. A typical human cochlea includes approximately two and a half helical turns of its various constituent channels. The cochlear lead (190) is inserted into one of the scalae, typically the scalae tympani (420), to bring the individual electrodes into close proximity with the tonotopically organized nerves.

The illustrative cochlear lead (190) includes a lead body (445). The lead body (445) connects the electrode array (195) to the internal processor (185, FIG. 3). A number of wires (455) pass through the lead body (445) to bring electrical signals from the internal processor (185, FIG. 3) to the electrode array (195). According to one illustrative example, at the junction of the electrode array (195) to the lead body (445) is a molded silicone rubber feature (450). The feature (450) can serve a variety of functions, including, but not limited to, providing a structure which can be gripped by an insertion tool, providing a visual indicator of how far the cochlear lead (190) has been inserted, and securing the electrode array (195) within the cochlea.

The wires (455) that conduct electrical signals are connected to the electrodes (465, 470) within the electrode array (195). For example, electrical signals which correspond to a low frequency sound may be communicated via a first wire to an electrode near the tip (440) of the electrode array (195). Electrical signals which correspond to a high frequency sound may be communicated by a second wire to an electrode (465) near the base of the electrode array (195). According to one illustrative example, there may be one wire (455) for each electrode within the electrode array (195). The internal processor (185, FIG. 3) may then control the electrical field generated by each electrode individually. For example, one electrode may be designated as a ground electrode. The remainder of the electrodes may then generate electrical fields which correspond to various frequencies of sound. Additionally or alternatively, adjacent electrodes may be paired, with one electrode serving as a ground and the other electrode being actively driven to produce the desired electrical field.

According to one illustrative example, the wires (455) and portions of the electrodes (470) are encased in a flexible body (475). The flexible body (475) may be formed from a variety of biocompatible materials, including, but not limited to medical grade silicone rubber. The flexible body (475) secures and protects the wires (455) and electrodes (465, 470). The flexible body (475) allows the electrode array (195) to bend and conform to the geometry of the cochlea.

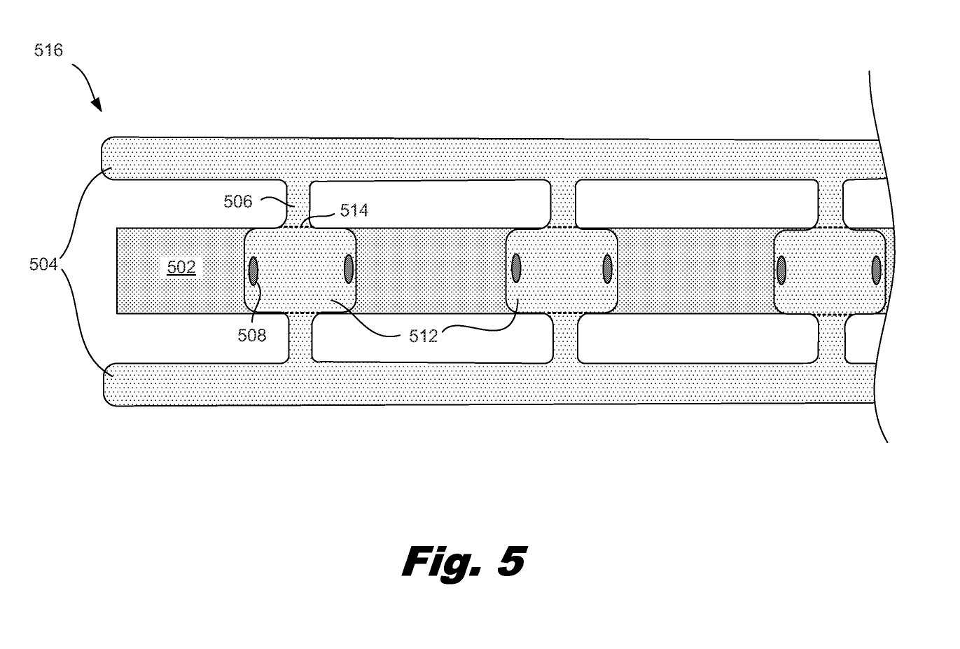

FIG. 5 is a diagram of a material which exhibits high charge transfer to cochlear tissues. This material has been formed into a tethered set of electrode pads (516) and attached to an underlying sacrificial substrate (502). As used in the specification and appended claims, the term “forming” or “formed” includes a wide variety of subtractive, additive, or transformative processes, including but not limited to, mechanical removal of material, laser cutting, electrical discharge machining (EDM), photolithographic techniques and etching, electron beam machining, abrasive flow machining, casting, extruding, stamping, imprinting, molding, and other suitable processes. According to one illustrative example, a number of generally rectangular electrode pads (512) have been formed along the center of the patterned high charge transfer material. The electrode pads (512) may have a number of other shapes including, but not limited to circular, oval, square, or trapezoidal. Further, the shape and size of the electrode pads may vary throughout the tethered set (516). In some examples, it may be desirable to form the sheet into shapes with at least some three dimensional curvature.

Each electrode pad (512) is tethered to rails (504) by two tethers (506). As used in the specification and appended claims, the term “tether” or “tethered” refers to a connection between an electrode and the structure that holds the electrodes in a fixed spatial relationship with other electrodes. Ordinarily, the tether (506) has a relatively small cross-section compared to the electrode pad (512) and connects the perimeter of the electrode pad (512) and the rails (504). The tethers (506) can hold the electrode pads (512) rigidly in place to completely fix the electrode spacing or semi-rigidly such that they are close to their final spacing and can be put into an alignment fixture to adjust the final spacing. In one example, tether widths are between 50 and 250 microns and lengths of the tethers are between 100 and 500 microns. According to one illustrative example, the electrode pads (512) and tethers (506) are formed from a single sheet of high charge transfer material.

The high charge transfer material may be patterned using a number of techniques including, but not limited to, short pulse laser micromachining techniques. As used in the specification and appended claims, the term “short pulse” means pulses less than a nanosecond, such as in the femtosecond to hundreds of picosecond range. A variety of lasers can be used. For example, very short pulse laser machining may be performed using a picosecond laser, at UV, visible, or IR wavelengths. These very short pulse lasers can provide superior micromachining compared with longer pulse lasers. The very short pulse lasers ablate portions of the material without significant transfer of heat to surrounding areas. This allows the very short pulse lasers to machine fine details and leaves the unablated material in essentially its original state.

The set (516) of tethered electrode pads (512) is fixed to a sacrificial substrate (502). According to one illustrative example, the sacrificial substrate (502) may be an iron strip which is approximately the width of the electrode pads (512) and at least as long as the tethered set (516) of electrode pads. The tethered set (516) of electrode pads may be attached to the sacrificial substrate (502) in a variety of ways, including resistance welding or laser welding. One or more weld joints (508) can be made for each electrode pad (512). The spacing of the electrode pads (512) is initially maintained by the tethers (506). The tethers (506) are cut after the welds (508) are formed. According to one illustrative example, the tethers (506) are cut at or near the dotted lines (514). After the tethers (506) are cut, the iron strip (502) maintains the desired electrode pad (512) spacing and orientation.

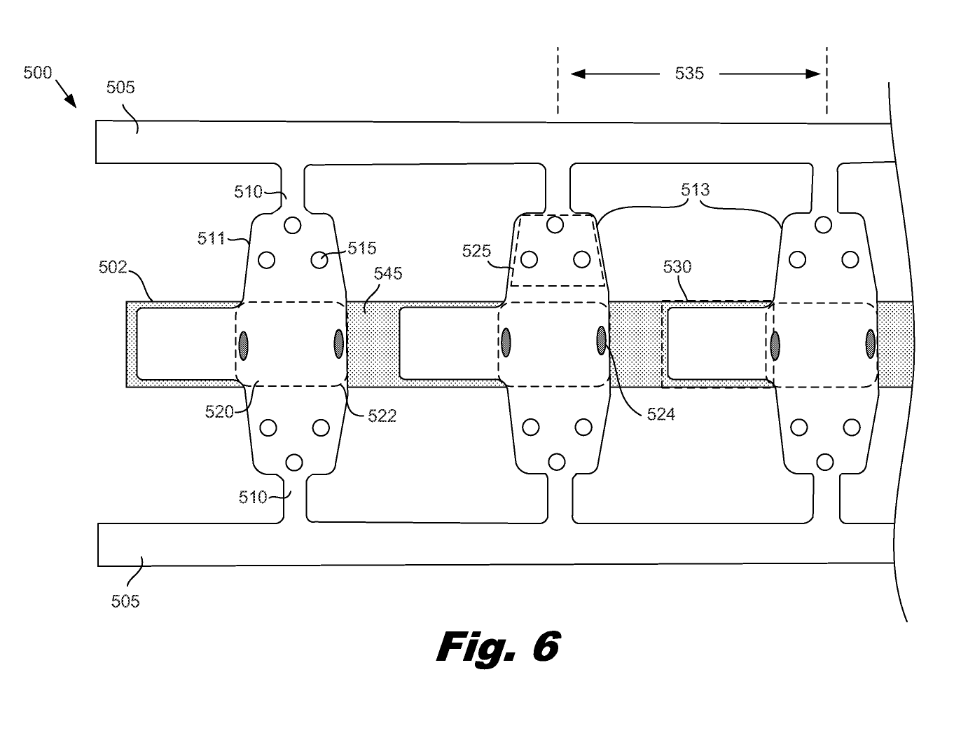

FIG. 6 is a diagram of an illustrative tethered set (500) of electrode assembly support structures with lateral wings (513) which have been machined from a flexible electrically conductive material. As used in the specification and appended claims, the term “flexible material” or “flexible electrically conductive material” refers to a material with a thickness of 20 to 1000 microns which can be creased or folded at greater than 90 degree angles without significant cracking or other failure at the crease or fold. For example, some platinum and platinum alloys are flexible materials according to this definition. According to one illustrative example, the tethered set (500) of electrode assembly support structures (513) with lateral wings (525) is machined from a platinum or platinum alloy foil using short pulse laser machining. For example, the sheet material may be between 20 and 50 micron thick platinum or platinum alloy (such as platinum/iridium having up to 20% iridium).

As discussed above, after the tethers (506, FIG. 5) have been cut from the electrode pads (512, FIG. 5), the electrode pads remain fastened to the sacrificial substrate (502). The tethered set (500) of support structures is aligned over the electrode pads so that a base portion (520) overlies each electrode pad (512, FIG. 5). The position of the underlying electrode pads is illustrated by the dashed line (522). A variety of methods could be used to connect the tethered set (500) of support structures to the electrode pads (512), including resistance or laser spot welding.

The dashed trapezoid illustrates the wing portions (525), which will be folded up to contain the wires. The wings (525) may have several additional features, such as holes (515). According to one illustrative example, during a later manufacturing step, a fluid matrix such as liquid silicone rubber is injected into a mold which contains the electrodes and their associated wiring. The fluid matrix flows through the holes (515), and then cures to form the flexible body. The holes (515) provide a closed geometry through which the fluid matrix can grip the electrode assembly.

A second dashed rectangle outlines a flap (530), which will be folded over a wire and welded to mechanically secure it to the electrode. This wire provides electrical energy to the electrode. The spacing (535) of the support structures (513) along the rails (505) matches the pitch of the underlying electrode pads (512, FIG. 5). The pitch of the electrode pads (512, FIG. 5) and the support structures (513) is also the pitch of the completed electrode assemblies in the final electrode array.

One or more welds (524) are made to join each of the support structures (513) to the underlying electrode pads (512, FIG. 5). A thin coating of silicone or other biocompatible insulating material can be deposited over an inner surface of the electrodes and wings and cured. This silicone layer provides a compliant and electrically insulating layer between the wires and the electrodes. The silicone layer can prevent mechanical abrasion and/or electrical shorting of the wires. According to one illustrative example, the wires are also individually insulated. For example, the wires may be individually insulated by a parylene coating. The tethers (510) are then cut and the tethers and rails (505) are removed.

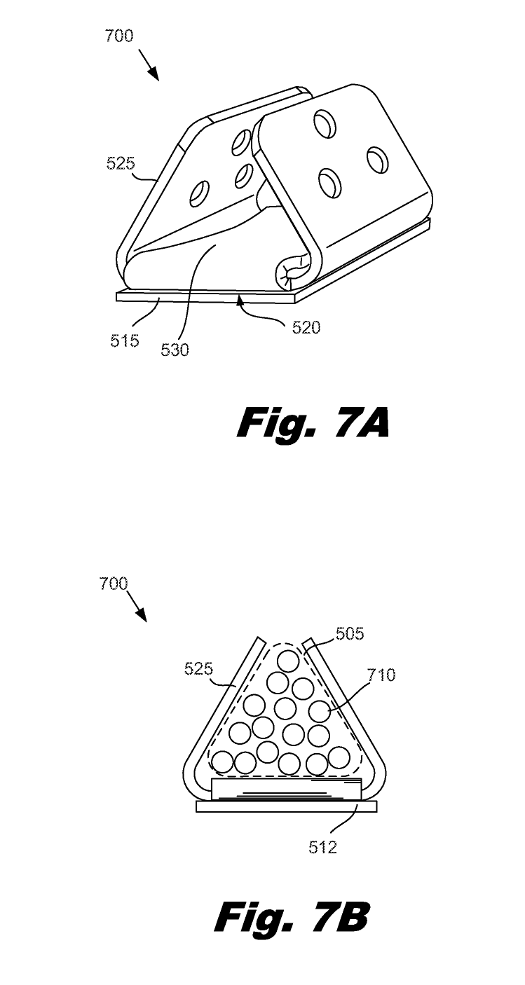

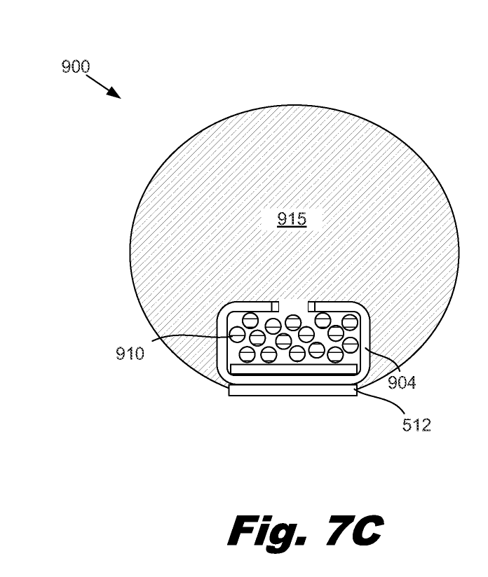

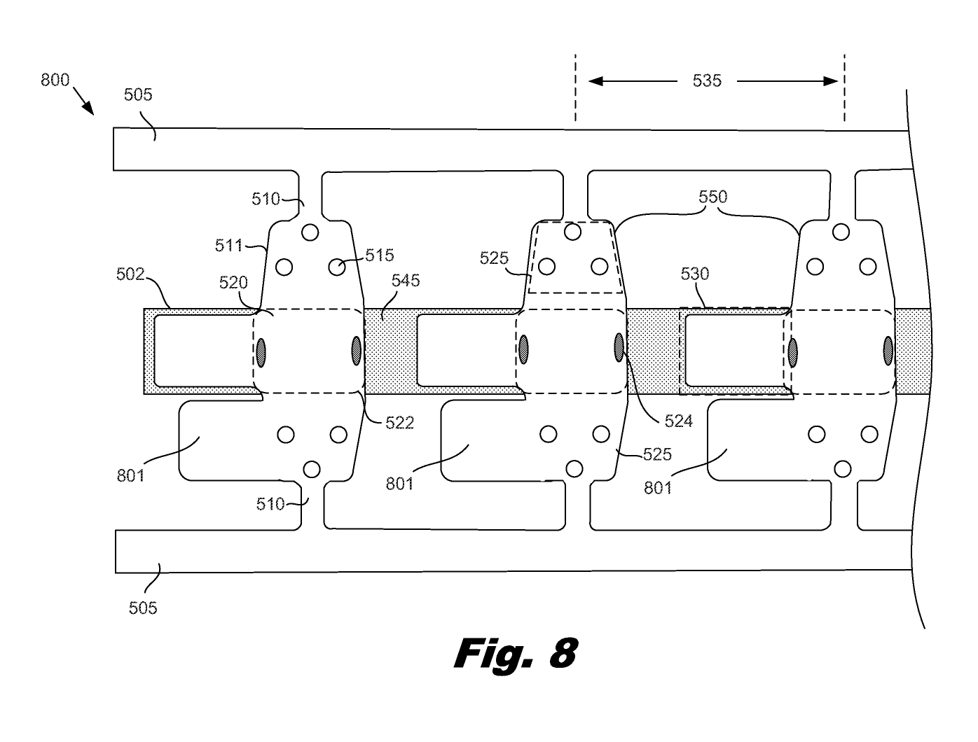

FIG. 7A is a perspective view of another illustrative example of a composite electrode assembly (700), which includes an integral wire carrier and an electrode pad (512) welded on the bottom of the folded support structure. For clarity of illustration, the wires are not shown in FIG. 8A. As discussed above, the flap (530) is folded over the wire associated with this composite electrode assembly (700) and welded to electrically and mechanically secure it in place. The wings (525) are folded up to secure the wires for the more distal electrodes and form a bundle of wires which passes back along the electrode array to the cochlear lead and to the internal processor. The electrode pad (512) is on the underside (520) of the folded support structure (530). The electrode pad (512) is not covered by the flexible body and is consequently exposed to the body tissues and fluids within the cochlea. The activated surface of the electrode pad (512) transfers electrical charge from the connected wire to the tissues. As discussed above, the electrode pad (512) may be formed from a variety of materials. According to one illustrative example, the electrode pad (512) has an activated iridium oxide layer on its external surface. The activated iridium oxide layer may have a charge transfer capability of approximately 3 to 7 mC/cm̂2. This charge transfer is significantly greater than a smooth platinum surface which typically has a charge transfer capability of approximately than 1 mC/cm̂2. The transferred charge creates an electrical field through the surrounding tissues, thereby stimulating the adjacent auditory nerve.

FIG. 7B is a cross-sectional view of the composite electrode assembly (700) shown in FIG. 7A. Cross-sections of the wires (710) are shown in a wire bundle (805) contained by the wings (525). As discussed above, this wire bundle (805) passes through the entire length of the electrode array (195, FIG. 3); however, each individual wire within the bundle terminates at the electrode to which it is welded.

FIG. 7C is a cross-sectional view of a different example of a composite electrode assembly (900). In FIGS. 7A and 7B, the wings are folded so that the resulting wire carrier is triangular. However, the wings may also be folded to create a rectangular wire carrier (904) as shown in FIG. 7C. As with other examples, the wires (910) are held by the wire carrier (904). FIG. 7C also shows the wire carrier (904) partially embedded in the flexible material (915) that constitutes the lead body.

A single lead may include some wire carriers of a triangular shape as shown in FIG. 7B and of a rectangular shape as shown in FIG. 7C. Different wire carrier shapes may be better suited for different locations along the lead.

During implantation and during operation of a cochlear lead, it is advantageous if the cochlear lead does not twist around its longitudinal axis. Such twisting can, in various examples, displace electrodes, cause tissue damage and place undesirable torsional stress on the lead itself. Twisting may also orient the electrode contacts away from the neural elements to be stimulated. Consequently, various principles and examples are described below for forming a cochlear lead that resists twisting about its longitudinal axis, this longitudinal axis being defined as a line running lengthwise along the center of the cochlear lead.

As will be described in greater detail below, an electrode assembly for a cochlear lead configured to stimulate an auditory nerve from within a cochlea, includes a conductive support structure for supporting an electrode and having two wings that are folded toward each other to form a wire carrier for a bundle of wires of the cochlear lead. At least one of the wings has at least one tab extending from that wing in a direction along a longitudinal axis of the cochlear lead to inhibit twisting of the cochlear lead.

The present specification also describes a cochlear lead having a flexible lead body; and a plurality of electrode assemblies partially embedded in the flexible body, the plurality of electrode assemblies being configured to stimulate an auditory nerve from within a cochlea. At least one of the electrode assemblies includes a conductive support structure for supporting an electrode and having at least two wings that are folded toward each other to form a wire carrier in which a bundle of wires is held. Not all the electrode assemblies need include the wings.

As used herein, “wire” refers generally to any conductive line for carrying an electrical signal. In various examples, a wire may be straight, have a zig-zag shape, be helically wound with other wires, be braided with other wires, individually insulated or not, of a single material or not, cabled or not. At least one wire is electrically connected to each electrode assembly along the flexible lead body. At least one of the wings has a tab extending from that wing in a direction along a longitudinal axis of the flexible lead body to inhibit twisting of the flexible lead body.

Additionally, the present specification describes a method of forming a cochlear lead including forming a plurality of electrode assemblies along a flexible lead body, the plurality of electrode assemblies being configured to stimulate an auditory nerve from within a cochlea. Forming each electrode assembly includes forming a conductive support structure having a base and two wings extending from the base, at least one of the wings comprising a tab extending from that wing along a longitudinal axis of the flexible lead body to inhibit twisting of the flexible lead body; and folding the two wings toward each other to form a wire carrier in which a bundle of wires is held, each wire connecting to a respective electrode assembly along the flexible lead body.

Similar to FIG. 6, FIG. 8 illustrates an illustrative tethered set (800) of electrode assembly support structures (550) with lateral wings (525) which have been machined from a flexible electrically conductive material. Unlike the set of winged support structures in FIG. 6, the support structures in FIG. 8 include both lateral wings and at least one tab (801) that extends from a wing (525). The at least one tab (801) extends longitudinally along the longitudinal axis of the set (800) and, eventually, the cochlear lead.

As noted above, during implantation and during operation, it is advantageous if a cochlear lead does not twist around its longitudinal axis. Such twisting can, in various examples, displace electrodes, cause tissue damage and place undesirable torsional stress on the lead itself. Consequently, one or more tabs (801), as illustrated in FIG. 8, are added to the wings (525) of electrode assembly structures. As will be shown below, these tabs provide a structure in the electrode assemblies that increases rigidity and resists twisting of the cochlear lead about its longitudinal axis.

FIG. 9 is a perspective view of one illustrative example of a composite electrode assembly having an integral wire carrier that resists twisting, according to one example of principles described herein. As discussed above, the flap (530) is folded over the wire associated with this composite electrode assembly and welded to electrically and mechanically secure it in place. The wings (525) are folded up to secure the wires for the more distal electrodes and form a bundle of wires which passes back along the electrode array to the cochlear lead and to the internal processor. (See FIG. 7B).

The electrode pad is on the underside (520) of the support structure (550). The electrode pad is not covered by the flexible body and is consequently exposed to the body tissues and fluids within the cochlea. The activated surface of the electrode pad transfers electrical charge from the connected wire to the tissues.

As discussed above, the tab (801) extends from a wing (525) along the longitudinal direction of the cochlear lead. This tab (801) or a number of such tabs in a variety of configurations increases the resistance to twisting of the cochlear lead both during implantation and during use.

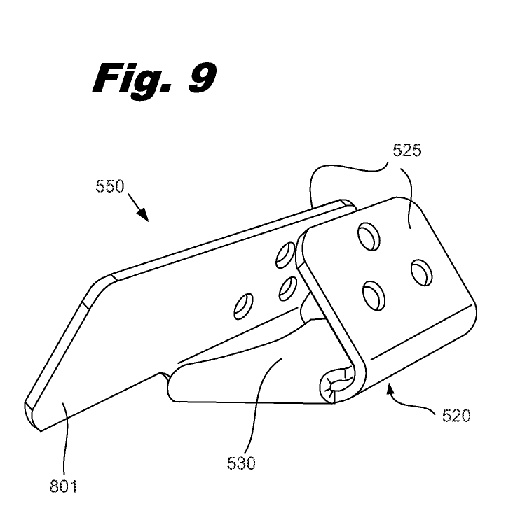

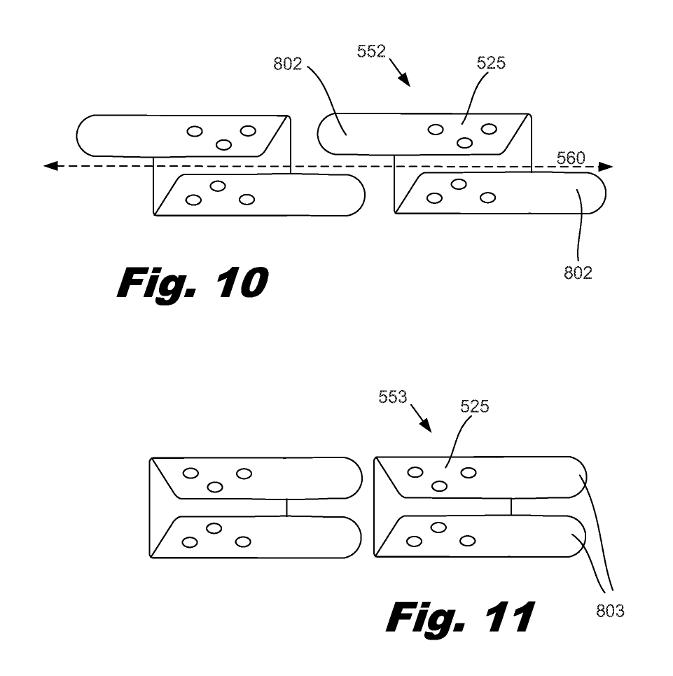

FIG. 10 is a top view of another illustrative example of a composite electrode assembly having an integral wire carrier that resists twisting, according to one example of principles described herein. In this example, the support structure (552) includes the opposing lateral wings (525) as in previous examples. However, each of the wings has a tab (802), the tabs (802) on the two wings (525) extending in opposite directions. Thus, both wings of the conductive support structure each comprise a tab extending from that wing in a direction along the longitudinal axis of the cochlear lead. The tabs are not on the longitudinal axis, but extend along the longitudinal axis.

In FIG. 10, a second support structure of the same type is shown to illustrate how successive electrode assemblies may lay along a cochlear lead. The tabs (802) will resist twisting of the cochlear lead about its longitudinal axis (560).

FIG. 11 is a top view of another illustrative example of a composite electrode assembly having an integral wire carrier that resists twisting, according to one example of principles described herein. In this example, the support structure (553) includes the opposing lateral wings (525) as in previous examples. However, each of the wings has a tab (803), the tabs (803) on the two wings (525) extending in the same direction.

In FIG. 11, a second support structure of the same type is shown to illustrate how successive electrode assemblies may lay along a cochlear lead. The tabs (803) will resist twisting of the cochlear lead about its longitudinal axis.

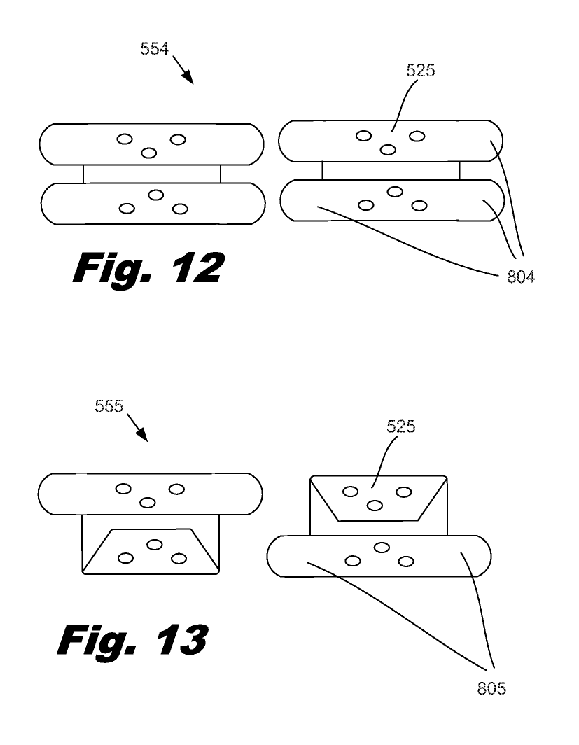

FIG. 12 is a top view of another illustrative example of a composite electrode assembly having an integral wire carrier that resists twisting, according to one example of principles described herein. In this example, the support structure (554) includes the opposing lateral wings (525) as in previous examples. However, each of the wings has a two tabs (804). The tabs (804) on the each wings (525) extend in opposite directions for a total of four tabs (804) on the wings (525) of this support structure (554).

In FIG. 12, a second support structure of the same type is shown to illustrate how successive electrode assemblies may lay along a cochlear lead. The tabs (804) will resist twisting of the cochlear lead about its longitudinal axis.

FIG. 13 is a top view of another illustrative example of a composite electrode assembly having an integral wire carrier that resists twisting, according to one example of principles described herein. In this example, the support structure (555) includes the opposing lateral wings (525) as in previous examples. However, only one of the wings has tabs (805). The tabs (805) on the wing (525) extending in opposite directions.

In FIG. 13, a second support structure of the same type is shown to illustrate how successive electrode assemblies may lay along a cochlear lead. As shown, the wing (525) with the tabs (805) may be on an alternating side of the row of support structures (55). The tabs (805) will resist twisting of the cochlear lead about its longitudinal axis.

Thus, for the examples shown in FIGS. 12 and 13, at least one of the wings has two tabs extending from that wing in opposite directions along the longitudinal axis of the flexible lead body to inhibit twisting of the flexible lead body. The other wing may also have one or more tabs or may have no tabs.

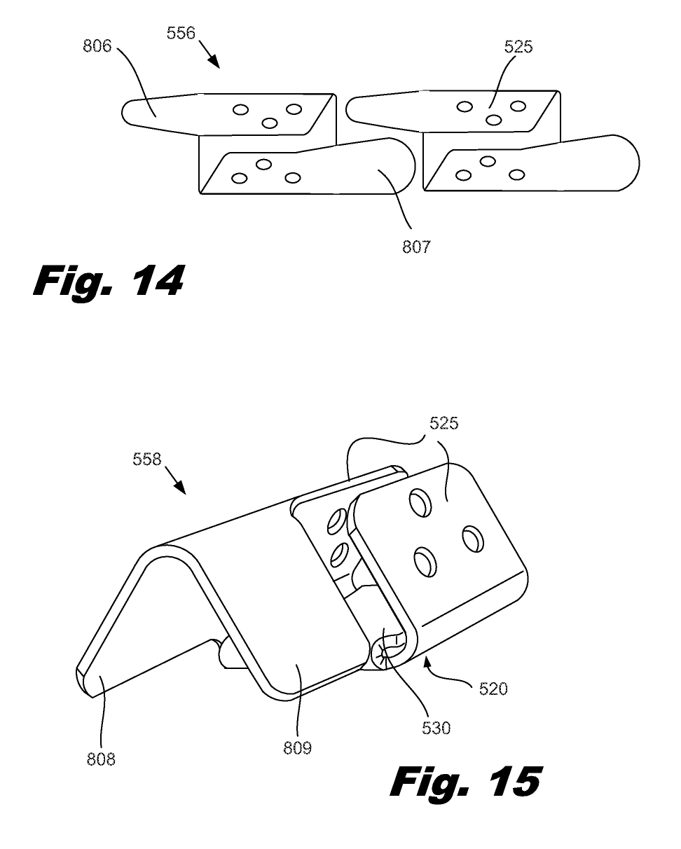

FIG. 14 is a top view of another illustrative example of a composite electrode assembly having an integral wire carrier that resists twisting, according to one example of principles described herein. In this example, the support structure (556) includes the opposing lateral wings (525) as in previous examples. Each of the wings has a tab (806. 807). However, the tabs (806, 807) are of different shapes and point in opposite directions. As shown, the tab (806) on one of the lateral wings (525) has a tapered shape, narrowing to a rounded tip in a direction away from the lateral wing on which it is disposed. The tab (807) on the opposite lateral wing extends in an opposite direction and expands in width to a rounded bulb in a direction away from the lateral wing on which it is disposed.

In FIG. 14, a second support structure of the same type is shown to illustrate how successive electrode assemblies may lay along a cochlear lead. As shown, the wings (525) with the tapering tabs (806) are on a common side of the longitudinal axis, which wings with the expanding and bulbous tabs (807) are together on an opposite side of the longitudinal axis. As in all other examples, the tabs (806, 807) will resist twisting of the cochlear lead about its longitudinal axis.

FIG. 15 is a perspective view of one illustrative example of a composite electrode assembly having an integral wire carrier that resists twisting, according to one example of principles described herein. The view shown in FIG. 15 is similar to that of FIG. 9. The flap (530) is folded over a wire associated with this composite electrode assembly and welded to electrically and mechanically secure that wire in place. The wings (525) are folded up to secure the wires for the more distal electrodes and form a bundle of wires which passes back along the electrode array to the cochlear lead and to the internal processor. (See FIG. 7B).

The electrode pad is on the underside (520) of the support structure (550). The electrode pad is not covered by the flexible body and is consequently exposed to the body tissues and fluids within the cochlea. The activated surface of the electrode pad transfers electrical charge from the connected wire to the tissues.

A tab (808) extends from a wing (525) along the longitudinal direction of the cochlear lead. This tab (808) includes a secondary tab (809) that extends from the main tab (808). As shown in FIG. 15, this secondary tab (809) is folded into the plane of the opposite wing (525) as that from to which it is connected. This extends the length of the wire carrier formed by the support structure (558), with a tab (808, 809) on two sides of the triangular wire-carrying pathway before the wires pass between the wings (525). As in all other examples, the tab (808) and the secondary tab (809) will resist twisting of the cochlear lead about its longitudinal axis.

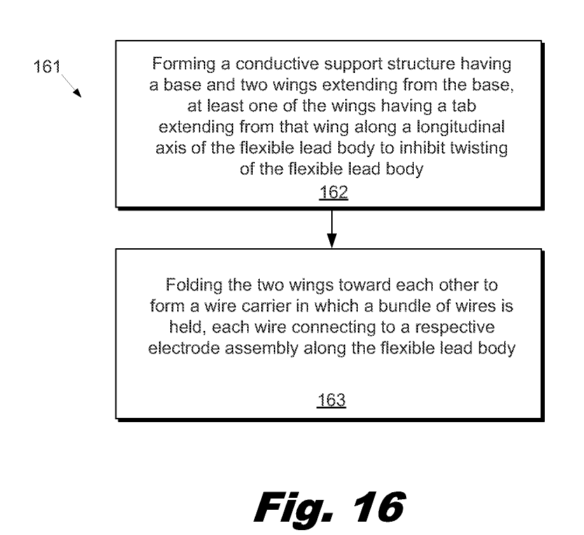

FIG. 16 is a flowchart showing one illustrative method for forming an electrode in a cochlear electrode array, according to one example of principles described herein. As illustrated, this method (161) includes forming (162) a conductive support structure having a base and two wings extending from the base, at least one of the wings having a tab extending from that wing along a longitudinal axis of the flexible lead body to inhibit twisting of the flexible lead body. The method then includes folding (163) the two wings toward each other to form a wire carrier in which a bundle of wires is held, each wire connecting to a respective electrode assembly along the flexible lead body.

The preceding description has been presented only to illustrate and describe examples and examples of the principles described. This description is not intended to be exhaustive or to limit these principles to any precise form disclosed. Many modifications and variations are possible in light of the above teaching.

1. An electrode assembly for a cochlear lead configured to stimulate an auditory nerve from within a cochlea, the electrode assembly comprising:
a conductive support structure for supporting an electrode and having at least one wing folded to form a wire carrier for a bundle of wires of the cochlear lead; and
at least one wing comprising a tab extending from that wing in a direction along a longitudinal axis of the cochlear lead to inhibit twisting of the cochlear lead.

2. The electrode assembly of claim 1,
wherein the conductive support structure comprises two lateral wings folded toward each other to form the wire carrier; and
wherein both wings of the conductive support structure each comprise a tab extending from that wing in a direction along the longitudinal axis of the cochlear lead to inhibit twisting of the cochlear lead.

3. The electrode assembly of claim 1, wherein at least one of the wings comprises two tabs extending from that wing in opposite directions along the longitudinal axis of the flexible lead body to inhibit twisting of the flexible lead body.

4. A cochlear lead comprising:
a flexible lead body; and
a plurality of electrode assemblies partially embedded in the flexible body, the plurality of electrode assemblies being configured to stimulate an auditory nerve from within a cochlea, at least one of the electrode assemblies comprising:
a conductive support structure for supporting an electrode and having two wings that are folded toward each other to form a wire carrier in which a bundle of wires is held, at least one wire being electrically connected to each electrode assembly along the flexible lead body; and
at least one of the wings comprising a tab extending from that wing in a direction along a longitudinal axis of the flexible lead body to inhibit twisting of the flexible lead body.

5. The cochlear lead of claim 4, wherein both wings of the conductive support structure each comprise a tab extending from that wing in a direction along the longitudinal axis of the flexible lead body to inhibit twisting of the flexible lead body.

6. The cochlear lead of claim 5, wherein the tabs on the wings extend in the same direction.

7. The cochlear lead of claim 5 wherein the tabs on the wings extend in opposite directions.

8. The cochlear lead of claim 4, wherein at least one of the wings comprises two tabs extending from that wing in opposite directions along the longitudinal axis of the flexible lead body to inhibit twisting of the flexible lead body.

9. The cochlear lead of claim 8, wherein both wings each comprise two tabs extending from that wing in opposite directions along the longitudinal axis of the flexible lead body to inhibit twisting of the flexible lead body.

10. The cochlear lead of claim 8, wherein each of two successive electrode assemblies along the flexible lead body each have a conductive support structure with a wing that comprises two tabs extending from that wing in opposite directions along the longitudinal axis of the flexible lead body to inhibit twisting of the flexible lead body;
wherein the tabs of a first of the two successive electrode assemblies are on a wing on an opposite side of the first electrode assembly as are the tabs of a second of the two successive electrode assemblies so that tabs of the first of the two successive electrode assemblies do not overlap the tabs of the second of the two successive electrode assemblies.

11. The cochlear lead of claim 4, wherein the tab tapers to a narrower width in a direction away from the wing on which that tab is located.

12. The cochlear lead of claim 4, wherein the tab widens to a greater width in a direction away from the wing on which that tab is located.

13. The cochlear lead of claim 4, wherein the tab itself comprises a secondary tab that extends in a plane common to another of the wings than the wing on which that tab is located.

14. The cochlear lead of claim 4, wherein the support structure comprises a number of features to assist in securing the electrode in place.

15. The cochlear lead of claim 4, wherein the wings comprise a number of features on the wings to assist in electrically and mechanically attaching a wire to the electrode.

16. The cochlear lead of claim 4, wherein the wire carrier comprises a triangular opening formed by the two wings and a base of the conductive support structure, at least some of the wires passing through this triangular opening.

17. A method of forming a cochlear lead comprising forming a plurality of electrode assemblies along a flexible lead body, the plurality of electrode assemblies being configured to stimulate an auditory nerve from within a cochlea; wherein forming each electrode assembly comprises:
forming a conductive support structure having a base and two wings extending from the base, at least one of the wings comprising a tab extending from that wing along a longitudinal axis of the flexible lead body to inhibit twisting of the flexible lead body; and
folding the two wings toward each other to form a wire carrier in which a bundle of wires is held, each wire connecting to a respective electrode assembly along the flexible lead body.

18. The method of claim 17, wherein both wings of the conductive support structure each comprise a tab extending from that wing along the longitudinal axis of the flexible lead body to inhibit twisting of the flexible lead body.

19. The method of claim 17, wherein at least one of the wings comprises two tabs extending from that wing in opposite directions along the longitudinal axis of the flexible lead body to inhibit twisting of the flexible lead body.

20. The method of claim 17, further comprising forming the tab tapers to a narrower width or widens to a greater width in a direction away from the wing on which that tab is located.
